(19) United States
(12) Patent Application Publication
(10) Pub. No.: US 2010/0094423 A1
(43) Pub. Date: Apr. 15, 2010

(54) SYSTEMS AND METHODS FOR ASSESSMENT OF TENSION IN AN IMPLANT

(75) Inventors: Kevin T. FOLEY, Germantown, TN (US); Newton H. METCALF, Memphis, TN (US); Gary S. LINDEMANN, Memphis, TN (US); Virginia RICHARDSON, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/251,922

(22) Filed: Oct. 15, 2008

(51) Int. Cl. A61F 2/44; A61B 17/70

(52) U.S. Cl. 623/17.16; 623/17.11; 606/263

(57) ABSTRACT

Systems and methods for providing assessment of a tensioned state of a flexible implant are provided. The implant includes at least one marker having a first configuration when the implant is relaxed or not tensioned in a desired manner and a second configuration when the implant is tensioned as desired.

### BACKGROUND

Vertebral implants are often used in the surgical treatment of spinal disorders such as disc herniations, scoliosis, degenerative disc disease, and other curvature and degenerative abnormalities and fractures. Various types of treatments can be used. In some cases, spinal fusion is indicated to inhibit relative motion between vertebral members. In other cases, dynamic implants are used to preserve motion between vertebral members. For such treatments, flexible implants may be attached between two or more vertebrae in any one or combination of anterior, lateral, postero-lateral or posterior side applications to the vertebrae. In other procedures, flexible implants are positioned in the spinal disc space. In still other procedures, flexible implants are secured between at least two anatomical locations in a patient.

Flexible implants can be tensioned prior to final attachment between the anatomical locations for implantation and to provide the desired stabilization characteristic or other effect. Assessment of the amount of tension in the implant can be difficult to determine in view of the nature of the implant material, its implanted location, and other factors that may prohibit or inhibit assessment of the implant.

### SUMMARY

Systems and methods for assessment of a tensioned state of a flexible implant are provided. The implant includes at least one observable marker having a first configuration when the implant is relaxed or not tensioned in a desired manner and a second configuration when the implant is tensioned.

According to one aspect, an implant for implantation in a patient includes a flexible body with a relaxed state and a tensioned state where the tensioned state is for implantation between two implantation locations in the patient. The body includes a marker that has a first configuration in the relaxed state when observed from a first direction and a second configuration that differs from the first configuration when the body is in the tensioned state and observed from the first direction.

In another aspect, an implant for implantation in a patient includes a flexible body with a first configuration between opposite ends of the body when in a relaxed state and a second configuration between the opposite ends when in a tensioned state. The tensioned state is for securement between two implantation locations in the patient. The body includes an elongated marker that forms a non-linear band when observed from a first direction and the body is in the first configuration and a straight band when observed from the first direction and the body is in the second configuration.

In another aspect, an implant for implantation in a patient includes a body formed from flexible material for implantation between locations in the patient. The body includes first and second elongated markers with a first configuration when observed from a first direction and with the body in a relaxed state. The first and second markers include a second configuration when observed from the first direction and the body is in a tensioned state. The second configuration provides an indication of the tensioned state to secure the body between the locations.

According to another aspect, a method for securing an implant in a patient comprises: providing the implant with a flexible body including at least one marker that is observable from a first direction relative to the body; securing the body to a first anatomical location in the patient; tensioning the body toward a second anatomical location; observing a configuration of the at least one marker from the first direction when the body is tensioned; comparing the observed configuration to a desired configuration; and securing the body to the second anatomical location when the observed configuration corresponds to the desired configuration.

These and other aspects are further discussed below.

### BRIEF DESCRIPTION OF THE FIGURES

FIGS. 1A and 1B show diagrammatic plan views of one embodiment flexible implant in a relaxed or non-tensioned configuration and tensioned configuration, respectively.

FIGS. 2A and 2B show diagrammatic plan views of another embodiment flexible implant in a relaxed or non-tensioned configuration and tensioned configuration, respectively.

FIGS. 3A and 3B show diagrammatic elevation views of one embodiment implantation procedure for the flexible implant.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

Flexible implants are provided from implantation between at least two locations in a patient in a body to provide repair, stabilization, replacement or other function within the body. The flexible implants include a relaxed state prior to implantation and a tensioned state that is desired for securement to the implantation locations in the patient. The body includes at least one marker having a first configuration when observed from a first direction when the body is in a relaxed state. The marker includes a second configuration when observed from the first direction when the body is in the tensioned state to provide an indication to the surgeon that the body is ready for securement to the implantation locations. As used herein, the relaxed state of the implant can include a state where no tension is applied to the implant, or a state where at least some tension is applied to the implant but the applied tension does not achieve a desired tension state.

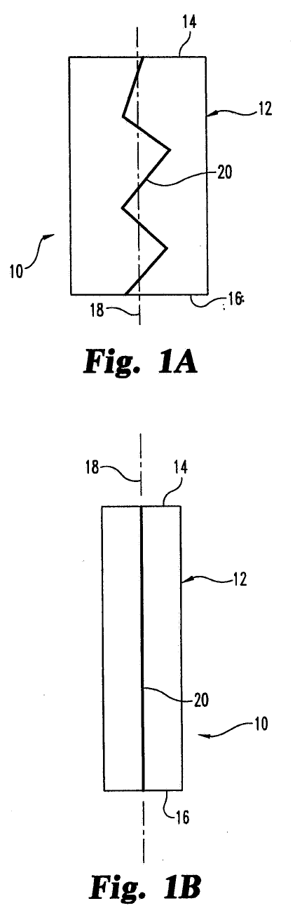

Various configurations for the implant and at least one marker are contemplated. For example, in FIG. 1A there is shown an implant 10 with a flexible body 12 extending between a first end 14 and an opposite second end 16 along a longitudinal axis 18. Body 12 also includes a marker 20 extending therealong between first and second ends 14, 16. In FIG. 1A, body 12 is in a relaxed state and marker 20 has a non-straight configuration along body 12. In FIG. 1B, body 12 is tensioned and marker 20 is reconfigured to have a straight configuration along body 12. The straight configuration provides an indication to the surgeon that body 12 is suitably tensioned for implantation between the locations in the patient. Marker 20 is observed from the same direction with body 12 in both the relaxed and tensioned states so that a comparison of the relative configurations of marker 20 can be readily made by the surgeon during the procedure.

In FIG. 1A the non-straight configuration of marker 20 is provided by a zigzagged band extending between opposite end 14, 16. Other embodiments contemplate other non-straight or non-linear configurations. For example, marker 20 can form a wave or series of compoundly curved segments along body 12. Marker 20 is also shown centered or approximately centered on longitudinal axis 18. Non-centered and offset relationships to longitudinal axis 18 are also contemplated. In another embodiment, marker 20 extends across the width of body 12 orthogonally to longitudinal axis 18. In a further embodiment, marker 20 is obliquely oriented to longitudinal axis 18 in one or both of the relaxed state and tensioned state. It is also contemplated that multiple markers could be provided on body 12.

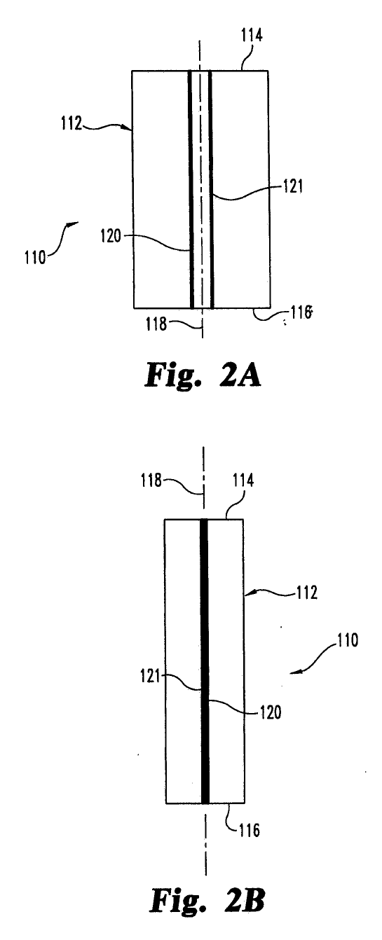

FIGS. 2A and 2B show another embodiment implant 110. Implant 110 includes a flexible body 112 extending between a first end 114 and an opposite second end 116 along a longitudinal axis 118. Body 112 also includes a first marker 120 and a second marker 121 extending therealong between first and second ends 114, 116. In FIG. 2A, body 112 is in a relaxed state and markers 120, 121 each has a straight configuration along body 112. Furthermore, markers 120, 121 are spaced parallel from one another and parallel to longitudinal axis 118. In FIG. 1B, body 112 is tensioned and markers 120, 121 are re-configured by being drawn toward one another in an overlapping arrangement along body 112. The overlapping configuration provides an indication to the surgeon that body 112 is suitably tensioned for implantation between the locations in the patient. Markers 120, 121 are observed from the same direction with body 112 in both the relaxed and tensioned states so that a comparison of the relative configurations of markers 120, 121 can be readily made by the surgeon during the procedure.

In FIGS. 2A-2B, markers 120, 121 include a straight configuration between opposite ends 114, 116 and are centered on or relative to longitudinal axis 118 in both the relaxed and tensioned state. Other embodiments contemplate non-centered relationships with longitudinal axis 118. Still other embodiments contemplate zigzagged, non-straight or non-linear configurations in one or both the relaxed and tensioned states. In another embodiment, markers 120, 121 extend across the width of body 112 orthogonally to longitudinal axis 118. In a further embodiment, markers 120, 121 are obliquely oriented to longitudinal axis 118 in one or both of the relaxed state and tensioned state.

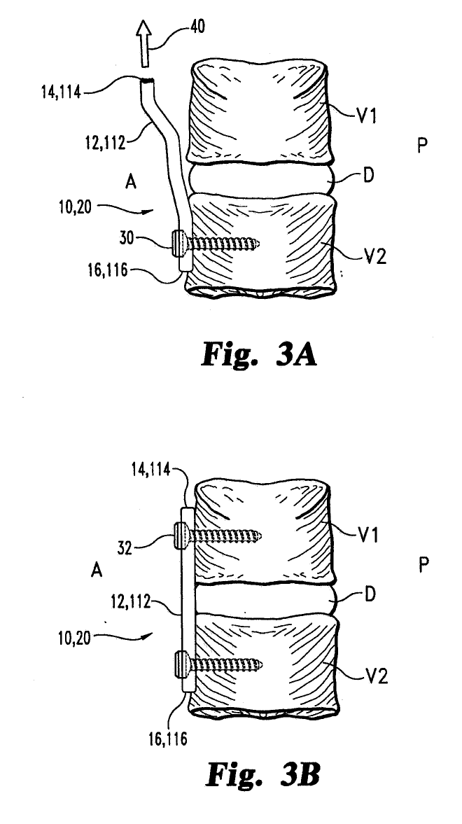

Implants 10, 110 each are shown with a body 12, 112 that is elongated and, as shown in FIG. 3B, flat to form of a sheet or strip of material. Other embodiments contemplate body 12, 112 is in the form of a cord or tubular member that is rounded around longitudinal axis 18, 118. In still other embodiments, body 12, 112 includes a non-uniform width along longitudinal axis 18, 118 to adapt it to the anatomy in which it is to be secured or to provide a desired stabilization characteristic.

It is also contemplated that bodies 12, 112 of implants 10, 110 can be made from elastic material and/or stretches when tensioned. In each of FIGS. 1A and 2A, body 12, 112 includes a first length between ends 14, 16 and ends 114, 116, respectively. In FIGS. 1B and 2B, body 12, 112 is stretched so that it lengthens to a second length between ends 14, 16 and ends 114, 116, respectively. In addition to lengthening the respective body 12, 112, the stretching of the material causes the markers 20, 120, 121 to reconfigure from their respective relaxed configurations relative to the body to another configuration when tensioned. Markers 20, 120, 121 are configured so that when the desired amount of tension is applied to body 12, 112, the markers 20, 120, 121 have a readily identifiable configuration when observed that indicates the desired tension has been obtained. For example, with respect to the embodiment of FIGS. 1A-1B, marker 20 forms a linear or straight band along the length of body 12 when the desired tension has been obtained. In FIGS. 2A-2B, markers 120, 121 overlap one another by touching, being located more adjacent to one another, or in alignment with one another when the desired tension has been obtained. During the surgical procedure, the surgeon can readily observe the markers 20, 120, 121 and their respective configurations so that implants 10, 110 can continue to be tensioned until the desired tension is obtained as indicated by the markers 20, 120, 121.

In still other embodiments, body 12, 112 is not elastic but is flexible so that it assumes a relaxed state and can be tensioned so that it is taut between its ends, and in the tensioned state its length is greater between its ends than when relaxed. In one specific embodiment, the flexible body can stretch so that it lengthens but is inelastic or partially inelastic so that is does not return to its relaxed state when the tension is released. In another specific embodiment, the flexible body is not readily stretched when tensioned but has a substantially fixed length between its ends when tension is applied thereto.

Markers 20, 120, 121 can be made from the same material as the respective body 12, 112 but provided with any suitable means to allow the marker to be observed and distinguished from body 12, 112 by any one or combination of naked eye visualization, x-ray imaging, fluoroscopy, CT scan, MRI imaging, endoscopic, microscopic, or other suitable viewing system or means. In one embodiment, markers, 20, 120, 121 include a color that contrasts with the color of the adjacent portions of body 12, 112 so the markers can be readily distinguished when observed. In another embodiment, body 12, 112 is comprised of radiolucent material and markers 20, 120, 121 are comprised of radiographic material or material with radiographic coatings, particles or segments that can be readily distinguished when observed. In still another embodiment, body 12, 112 is radiographic and markers 20, 120, 121 are radiolucent to provide a negative image of the marker configuration. In one specific embodiment, body 12, 112 is made from woven fibers, and markers 20, 120, 121 are contrasting fibers that are interwoven with the fibers of the body. In another specific embodiment, markers 20, 120, 121 are made from a layer or coating of contrasting color or radiographic material or particles applied to one or more surfaces of body 12, 112.

FIGS. 3A and 3B show one embodiment of a surgical procedure for implantation of implant 10, 110 between implantation locations in a patient. In the illustrated embodiment, the implantation location includes vertebrae V1 and V2 with disc space D therebetween. Implant 10, 110 is applied to an anterior side A of vertebrae V1, V2 to replicate or replace the anterior longitudinal ligament. Other embodiments contemplate placement of implants 10, 110 at posterior side P, or laterally, antero-laterally, postero-laterally or in combinations of such locations along the vertebrae. Furthermore, vertebrae V1 and V2 can be any one or more vertebrae in one or more of the cervical, thoracic, lumbar, or sacral regions of the spinal column. Also contemplated but not shown are placement of a spinal fusion or motion preserving implant in disc space D in conjunction with the stabilization provided by implant 10, 110. In still other procedures, implant 10, 110 can be placed at other non-spinal locations in the patient and secured between two or more implantation locations.

In FIG. 3A implant 10, 110 is secured to vertebra V2 with fastener 30 adjacent second end 16, 116. Implant 10, 110 is in a relaxed state so that marker 20 and markers 120, 121 are in a first configuration as shown and discussed above with respect to FIGS. 1A and 2A, respectively. A tension force is applied to first end 14, 114 as indicated by arrow 40 to move implant 10, 110 toward a tensioned state. Tension is increased until marker 20 and markers 120, 121 are in a second configuration that indicates the desired amount of tension has been applied, as discussed above with respect to FIGS. 1B and 2B. When the desired tension has been applied, implant 10, 110 is secured to vertebra V1 with a second fastener 32, as shown in FIG. 3B, to maintain implant 10, 110 in the tensioned state while it is secured between vertebrae V1 and V2. The placement and location of the implant 10, 110 relative to the vertebrae can also be checked and confirmed by determining the location of markers 20, 120, 121 relative to the vertebral anatomy, such as the midline, to confirm appropriate implant positioning.

Other procedures contemplate attachment of implant 10, 110 to more than two vertebrae or two vertebrae with one or more vertebrae therebetween. In the illustrated embodiment, fasteners 30, 32 are bone screws that extend through body 12, 112. It is contemplated that fasteners 30, 32 can include any suitable fastening mechanism, including uni-axial screws, multi-axial screws, hooks, clamps, rivets, interference anchors, suture anchors, plates, staples, wires, bands, tacks, adhesives, and interbody devices between vertebrae, for example. The fasteners can extend through implant 10, 110 or implant 10, 110 can be received in an eyelet, receiver, saddle, or other structure of the fastener and secured therein with a set screw, nut, cap, plug, tack, rivet, weld, locking mechanism, or other suitable securing structure.

Implants 10, 110 have application as a surgical orthopedic device that provides advantageous properties to treat bone defects. The device can be used to treat a variety of bone defects including diseased, damaged, and/or fractured bone. The defective bone structures can be the result of damaged, traumatized, and/or diseased bone tissue. Implants 10, 110 can also be employed in the treatment of scoliosis and/or kyphosis. Furthermore, by use of the term “orthopedic device”, it is intended to include within its meaning a device or implant that can be used to treat or repair defective, diseased, and/or damaged tissue of the muscular/skeletal system(s) and can include attaching bone portions together, reinforcing a single unitary bone portion and/or attaching ligaments to one or more bone portions. Furthermore, the devices and methods described herein can be used to treat any type of bone or related tissue including, without limitation, articulating bone and bone joints, long bones, short bones, flat bones, cortical bone tissue, cancellous bone tissue and associated ligaments.

Implants 10, 110 can be fabricated and/or composed of suitable material tailored to treat and repair a variety of muscular/skeletal defects and disorders. Physical characteristics and properties of implants 10, 110 and associated components such as tensile strength, elasticity or stiffness and creep can be varied as desired. In one embodiment, implants 10, 110 are provided to have a tensile strength sufficient to restrain or maintain the attached bone pieces or portions in a desired orientation and/or spacing with each other despite the biomechanical stresses exerted by the muscular/skeletal system during normal activity. The elasticity or stiffness of implant 10, 110 can also be varied for a particular application or treatment. In addition or in the alternative, implants 10, 110 can deform or creep under strain.

Implants 10, 110 can be provided in a variety of sizes, cross-sections, lengths, widths, and shapes. Implants 10, 110 can be substantially cylindrical or a flat, ribbon-like configuration, whether formed of a single fiber or filament or a plurality of fibers or filaments. When provided as a plurality of fibers, the fiber can be arranged and/or fashioned as desired including without limitation, braiding, wounding, parallel, twisting, and weaving (either 2 dimensional or 3 dimensional weaves). Bodies 12, 112 can be provided in multiple layers stacked one upon the other, or in one or more interwoven layers.

Various suitable materials for implants 10, 110 are contemplated, including, without limitation, degradable or resorbable polymeric materials, metal materials, tissue materials, non-resorbable polymeric material, ceramic material, shape memory material, and composites thereof. Implants 10, 110 can also be coated or impregnated with anti-adhesive material that will prevent tissue and vasculature from attaching thereto.

In one specific embodiment, bodies 12, 112 are made from a soft fiber material. Soft fiber material can include polymeric material, such as SPECTRA fiber, nylon, carbon fiber and polyethylene, among others. In another embodiment, implants 10, 110 are made from commercially available ultra high molecular weight polyethylene (UHMWPE). Examples of suitable polymers include DACRON and GORE-TEX. In another embodiment, bodies 12, 112 are made from metal wire mesh. It is contemplated that the wire can be made from stainless steel, cobalt-chrome alloy, titanium, titanium alloy, or nickel-titanium, among others.

In selected embodiments, implants 10, 110 can be provided with markers 20, 120, 121 to exhibit suitable imaging characteristics including a specified radiopacity to enable the marker to be observed under common medical diagnostics imaging techniques. The radiopacity can help ascertain that the implant has been correctly placed and tensioned, and remains in place with the desired tension. In one form, the radiopacity can be provided by incorporating a radio-opaque element into body 12, 112. In one example, markers 20, 120, 121 include radio-opaque fibers or filaments are associated with body 12, 112. The fibers or filaments can be composed of a radio-opaque material such as a metal filament or a polymeric filament that has been impregnated or coated with a radio-opaque material such as a metallic material.

The radio-opaque markers can be provided in a variety of materials. Examples of radio-opaque materials that can be used in the present invention include, without limitation: nitinol, titanium, titanium-vanadium-aluminum alloy, cobalt-chromium alloy, cobalt-chromium-molybdenum alloy, cobalt-nickel-chromium-molybdenum alloy, stainless steel, tantalum, niobium, hafnium, tungsten, gold, silver, platinum, or iridium metals, alloys, and mixtures thereof. The radio-opaque element can be provided as one or more fibers, filaments or strands made from the above material or one or more fibers, filaments, or strands coated or impregnated with one or more of the materials listed above. In another embodiment, the fibers, filaments or strands are coated with barium sulfate. The radiopacity of markers 20, 120, 121 can be indefinite or can be selected to provide radiopacity for a shorter duration.

While the invention has been illustrated and described in detail in the drawings and foregoing description, the same is to be considered as illustrative and not restrictive in character, and that all changes and modifications that come within the spirit of the invention are desired to be protected.

What is claimed is:

1. An implant for implantation in a patient, comprising:
a flexible body that is movable between a relaxed state between opposite ends thereof and a tensioned state between said opposite ends, wherein said tensioned state is for implantation between at least two implantation locations in the patient, said body including a marker, wherein said marker includes a first configuration when observed from a first direction with said body in said relaxed state and a second configuration observed from said first direction that differs from said first configuration when said body is in said tensioned state.

2. The implant of claim 1, wherein said elastic material is radiolucent and said marker is radio-opaque.

3. The implant of claim 1, wherein said marker includes a first band extending along said length and a second band extending along said length, wherein when in said first configuration said first band is spaced from said second band and when in said second configuration said first and second bands overlap one another along said length.

4. The implant of claim 3, wherein when in said first configuration said marker forms a nonlinear band along said length and when in said second configuration said marker forms a linear band along said length.

5. The implant of claim 1, wherein when in said first configuration said marker forms a zigzagged band along said length and when in said second configuration said marker forms a straight band along said length.

6. The implant of claim 1, wherein said body is made from woven strands of radiolucent fibers and said marker includes at least one fiber of radio-opaque material interwoven with said radiolucent fibers.

7. The implant of claim 1, wherein said marker includes a color that contrasts with a color of said body.

8. The implant of claim 1, further comprising first and second fasteners for securing said body to the implantation locations in the body.

9. The implant of claim 1, wherein said marker extends from one of said opposite ends to the other of said opposite ends.

10. The implant of claim 1, wherein said body is made from an elastic material and in said relaxed state said body includes a first length between said opposite ends and in said tensioned state said body includes a second length between said opposite ends, and said second length is greater than said first length.

11. An implant for implantation in a patient, comprising:
a flexible body including a first configuration between opposite ends of said body when in a relaxed state and a second configuration between said opposite ends when in a tensioned state, wherein said tensioned state is for securement between two implantation locations in the patient, said body including an elongated marker that forms a non-linear band on said body when observed from a first direction and said body is in said first configuration and a straight band on said body when observed from said first direction and said body is in said second configuration.

12. The implant of claim 11, wherein when said body is in said first configuration said marker forms a zigzagged band along said first length.

13. The implant of claim 11, wherein said marker extends from one of said opposite ends to the other of said opposite ends of said body.

14. The implant of claim 11, wherein when in said first configuration said body includes a first length between said opposite ends and in said second configuration said body includes a second length between said opposite ends, and said second length is greater than said first length.

15. An implant for implantation in a patient, comprising:
a flexible body for implantation between locations in the patient, said body including first and second elongated markers, said first and second markers including a first configuration when observed from a first direction and said body is in a relaxed state and said first and second markers including a second configuration when observed from said first direction and said body is in a tensioned state, wherein said second configuration provides an indication of said tensioned state to secure said body between the locations.

16. The implant of claim 15, wherein said first and second markers are spaced from one another in said first configuration and said first and second markers overlap one another in said second configuration.

17. The implant of claim 16, wherein:
said body includes a length between opposite ends thereof and said opposite ends are spaced from one another in said second configuration for securement to respective ones of the locations; and
said first and second markers are straight bands extending along said length between said opposite ends of said body.

18. The implant of claim 17, wherein said first and second markers are parallel to one another in said first configuration.

19. The implant of claim 15, wherein:
in said relaxed state said body includes a first length between said opposite ends and in said tensioned state said body includes a second length between said opposite ends, said opposite ends beings spaced from one another in said tensioned state for securement to respective ones of the locations and said second length is greater than said first length.

20. A method for securing an implant in a patient, comprising:
providing the implant with a flexible body including at least one marker that is observable from a first direction relative to the body;
securing the body to a first anatomical location in the patient;
tensioning the body toward a second anatomical location;
observing a configuration of the at least one marker from the first direction when the body is tensioned;
comparing the observed configuration to a desired configuration; and
securing the body to the second anatomical location when the observed configuration corresponds to the desired configuration.

21. The method of claim 20, wherein observing the configuration includes observing a color contrast between the at least one marker and the body via naked eye visualization.

22. The method of claim 20, wherein observing the configuration includes observing the at least one marker via radiographic imaging.

23. The method of claim 22, wherein the body is radiolucent and the marker is radio-opaque.

24. The method of claim 20, wherein the first and second anatomical locations are first and second vertebrae of a spinal column and the body is positioned anteriorly along exterior surfaces of the first and second vertebrae.

25. The method of claim 24, wherein securing the body includes engaging the body to the first and second vertebrae with respective ones of first and second bone fasteners.

26. The method of claim 20, wherein the at least one marker includes a zigzagged configuration along the body when the body is in a relaxed state and the at least one marker is straight along the body in the desired configuration.

27. The method of claim 20, wherein the at least one marker includes a pair of elongated markers extending parallel to one another when the body is in a relaxed state and the elongated markers overlap one another in the desired configuration.

28. The method of claim 20, wherein the body includes a first length between opposite ends thereof in a relaxed state and a second length between the opposite ends thereof when the marker is in the desired configuration, the second length being greater than the first length.

29. The method of claim 28, wherein the at least one marker extends between the opposite ends of the body.

30. The method of claim 20, wherein the flexible body is elastic.
